(19) United States
(12) Patent Application Publication
(10) Pub. No.: US 2018/0161025 A1
(43) Pub. Date: Jun. 14, 2018

(54) DUAL ANCHORING DEVICE FOR THE SUSPENSION OF BIOACTIVE SUBSTANCES IMMEDIATELY FOLLOWING CESAREAN SECTION DELIVERY FOR CONTRACEPTION AND TREATMENT

(71) Applicant: PAT&CO BVBA, Lichtervelde (BE)

(72) Inventor: Dirk Wildemeersch, Ghent (BE)

(21) Appl. No.: 15/575,521

(22) PCT Filed: May 18, 2016
(86) PCT No.: PCT/EP2016/061128

§ 371 Date: Nov. 20, 2017

(30) Foreign Application Priority Data
May 20, 2015 (EP) 15168539.3

(51) Int. Cl. A61B 17/04

(52) U.S. Cl. CPC A61B 17/0401; A61F 6/208; A61B 17/0485

(57) ABSTRACT

The invention relates to an anchoring device specifically for the retention of bioactive substances in the immediate post-Cesarean section uterus for contraception. The invention includes a dual anchoring device, preferably a permanent anchoring knot, provided with a noose, for insertion in the top of the uterus with an inserter, and which is kept in place during the involution phase of the uterus by a supplementary means of temporary fixation, to overcome the risk of IUD/IUS dislodgment from the soft uterine wall. The anchoring device includes a biodegradable suture, consolidated with the permanent anchoring knot, for its temporary fixation to the serosa of the uterus. The anchoring knot can be easily removed from the uterus at any time after its insertion in the uterine wall and fixation to the serosa due to the fast biodegrading time of the suture.

### FIELD AND BACKGROUND OF THE INVENTION

Immediate postpartum insertion (within 10 minutes of delivery of the placenta) of an intrauterine device or system (IUD/IUS) is generally safe and effective. Studies have confirmed that postpartum IUD insertions, including those done immediately after placental delivery or cesarean section, do not result in increased morbidity. Compared with interval insertions, postpartum insertions do not increase the risk of infection, bleeding, uterine perforation or endometritis, nor do they affect the return of the uterus to its normal size.

Both copper and hormone-releasing IUD/IUS do not affect breastfeeding either, in contrast with systemic hormonal methods. Therefore, the IUD/IUS has a distinct advantage. The post-delivery period may also be a convenient time to have an IUD or an IUS inserted, since it may be one of the few times the woman is in contact with medical services.

In addition, IUDs do not require regular user compliance. Coital-dependent methods may be used inconsistently during the postpartum period by couples who think conception is less likely during this period. Also, if a woman says she wants no more children but has not had time to consider sterilization carefully, an IUD/IUS offers a reversible alternative.

The timing of insertion is important primarily because it influences the risk of expulsion. Expulsion can leave a woman unprotected from pregnancy without her realizing it. Ideally, postpartum insertion should take place within 10 minutes of placental delivery (immediate postplacental) or at about six weeks after birth, when a woman returns for a routine postpartum care visit. For women with limited access to medical care, the time of delivery offers a unique opportunity to address their need for contraception if the delivery takes place in a health-care center. The a priori hypothesis is that this practice is safe, but is associated with a higher expulsion rate than with interval insertion of the IUD.

As the main problem with postpartum insertions is the high expulsion rates of the IUD/IUS seen at that time which is much higher compared to that of interval insertions. The risk of expulsion is lower for insertions done within 10 minutes of delivery than for those done between 10 minutes and hospital discharge. The risk of expulsion can be as much as 40 or 50% which is unacceptably high. For this reason, the method never became popular and general use did not occur. In an attempt to reduce this risk, appropriate training in postpartum insertion techniques has been advocated but with minimum result. It appears, therefore, that the only solution to minimize the number of expulsions is to invent a means for attaching the IUD to the fundus of the uterus immediately post-partum.

Attachment of an IUD to the uterine fundus has been attempted using different techniques and has been the subject of a prior submission (PCT W091/00714). The anchoring system described in this patent was highly effective and reduced the expulsion rate substantially. However, it was difficult to remove the IUD, especially if required during the first few weeks or even months after insertion. The challenge remains therefore to develop and anchoring means which is easy to insert and remove.

### SUMMARY OF THE INVENTION

The present invention consequently is directed to an anchoring device, for its insertion and suspension of a copper-releasing device, or a hormone-releasing intrauterine system which can be inserted easily and safely, under direct vision, immediately after removal of the placenta and contraction of the uterus, and which can be removed within days after its insertion in case of a medical indication or urgency. This purpose is achieved by designing a suitable anchor which, when assembled together with the IUD or IUS, can be inserted and fixed quickly in the fundus of the uterus with a specially designed inserter as described previously.

There are several possible modes of realization. In this respect, we refer to previous submissions (U.S. Pat. No. 4,708.134 and U.S. Pat. No. 5,433,218). These patents describe the fixation of frameless copper and hormone-releasing IUDs in the uterus. This mode of realization is also possible for the suspension of active substances in the post-Cesarean section uterus. However, if used as such, the risk of expulsion of the IUD/IUS will still be unacceptably high because of the soft tissue of the wall of the uterus which is not compact enough to retain the small anchor.

Retention of the anchor in the soft tissue can be solved by adding material, i.e., biodegradable suture material to secure the position of the anchor in the soft tissue of the uterine fundus by temporarily attaching the anchor to the more resistant and dense tissue of the serosal layer of the uterus which covers the outside of the uterus. As the uterus involutes over weeks and regains its normal size, and the uterine tissue becomes denser, the permanent anchor will be sufficient to retain the IUD/IUS in situ while the temporary attachment suture degrades.

This aim is achieved by using the same anchoring principle as described previously but, in addition, by connecting the anchor to the serosa using a biodegradable suture. In case the permanent anchor consists of a surgical knot, a thin biodegradable suture could be attached to the knot and stitched to the serosa.

This characteristic of the invention is achieved by puncturing the uterus with an applicator immediately after removal of the placenta so that the anchor is visible at the surface of the uterus. The next step is to remove the applicator and to thread the noose of the anchoring knot with a biodegradable suture and fixing it to the serosa.

According to another characteristic of the invention, the anchor is a surgical knot, sufficiently small in diameter to allow easy removal of the attached device when necessary, even early after its insertion.

According to another characteristic of the invention, the anchor is a surgical knot with a small noose to allow threading with a biodegradable suture.

According to another characteristic of the invention, the biodegradable suture degrades completely within a few weeks with significant reduced tensile strength after only a few days or one week.

According to another characteristic of the invention, the biodegradable suture is fixed to the serosal layer of the uterus by stitching it to the serosa.

According to another characteristic of the invention, the biodegradable suture is fixed to the serosal layer of the uterus by attaching it with a metal or biodegradable clip.

According to yet another characteristic of the invention, IUD/IUS can be frameless or framed.

According to another characteristic of the invention, the hormone-releasing IUS consists of a rod or hollow tube with tail and anchoring means.

### DESCRIPTION OF THE DRAWINGS

The invention will be better understood with reference to the description as well as the attached drawings, which show, purely by way of example, various embodiments of the invention and in which:

FIG. 1 is a frameless IUD of which the anchor consists of a knot, which is inserted though the wall of the post-partum uterus, and which has a noose through which a biodegradable suture is threaded.

FIG. 2 and FIG. 3 show the fixation of the anchoring knot to the serosa of the uterus by stitching the suture to it or by fixing it to the serosa with a metal or biodegradable clip.

FIG. 4 is a representation of a hormone-releasing system with a fixation means attached to the top of the drug delivery system and with removal tail attached to the bottom of it.

FIG. 5 and FIG. 6 is a more detailed representation of the mode of realization to attach the anchoring means and the removal tail to the drug delivery rod described in FIG. 4.

FIG. 7 is a representation of a tube-shaped drug delivery compartment to which the anchoring means and removal tail, being a suture with anchoring knot on the top of the suture, is threaded through the lumen of the drug delivery tube and of which the tube is prevented from sliding off the anchoring suture my means of a metal or plastic clips.

### DESCRIPTION OF THE PREFERRED MODE OF REALIZATION

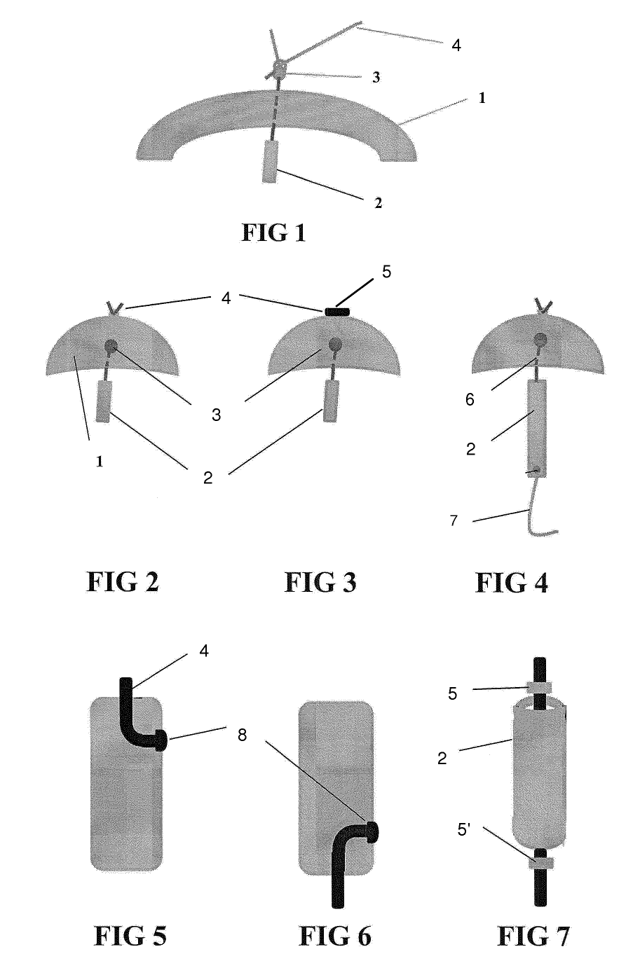

Referring to FIG. 1, showing the fundal part of the uterus 1 with IUD 2 and device anchor 3 which is inserted through the fundal wall and of which the biodegradable suture 4 is inserted through the noose of the anchoring knot 3.

FIG. 2 and FIG. 3 show a segment of the uterine fundus 1 with anchoring knot 3 and the preferred mode of realization consisting of temporarily attaching the anchoring knot to the serosa of the uterus; in FIG. 2 by tying the suture 4 to the serosa and in FIG. 3 by fixing the suture to the serosa with clip 5.

Referring now particularly to FIG. 4, an anchoring means 6 is integrated into the upper part of a drug delivery system 2 for its retention in the uterus, and is provided with a tail 7 also integrated into the drug delivery system for removal of the IUS.

FIG. 5 shows the preferred mode of realization of the attachment of the anchoring suture 4 to the drug delivery rod by spherical thermo-deformation of the end of the suture 8.

FIG. 6 shows a similar mode of realization of the attachment of the removal tail to the drug delivery by spherical thermos-deformation of the end of the tail.

FIG. 7 is a schematic representation of the fixation of a tube-shaped drug delivery system 2 by means of two metal clips 5 and 5′ onto the suture which keep the system affixed to the anchoring thread.

The invention has been described and illustrated merely by way of example which is in no way restrictive. Numerous changes in its conception may be made without departing from the spirit of the invention.

What is claimed is:

1. An anchoring device for easy and safe anchoring of an intrauterine device or intrauterine system to the top (fundus) of the post-Cesarean section uterus, said device comprising an anchoring knot, said knot comprising a body and a noose, wherein said noose allows threading of a biodegradable suture for temporary fixation of the anchoring device in the soft muscular tissue to prevent the intrauterine device or system from being expelled from the uterus.

2. The anchoring device according to claim 1 which comprises an anchoring knot threaded with a biodegradable suture, wherein the biodegradable suture is suitable to be tied to the serosa.

3. The anchoring device according to claim 1 which comprises an anchoring knot threaded with a biodegradable suture, wherein the biodegradable suture is suitable to be fixed to the serosa with a surgical metal or biodegradable clip.

4. A drug delivery compartment for insertion and anchoring to the fundus of the postpartum uterus consisting of a drug delivery body provided with an anchoring device according to claim 1 at the top of the drug delivery rod, and with a tail at the bottom for easy removal of the intrauterine device or intrauterine system from the uterine cavity.

5. The drug delivery compartment according to claim 4, for insertion and anchoring to the fundus of the postpartum uterus, wherein the intrauterine device or intrauterine system as well as the removal tail are integrated in top and the bottom of the drug delivery rod by threading a suture through a preformed curved channel in the rod and fixed in place by a thermoformed spherical thickening at the end of the suture.

6. A tube-shaped drug delivery compartment fixed on a suture thread comprising metal or plastic clips placed on the suture at least below the tube to keep the tube affixed to the anchoring thread.

7. A method for preventing the expulsion of an IUD from the post-Caesarian uterus, said method comprising the following steps:
immediately post-Cesarean, inserting an IUD in the uterus of a patient with an applicator, thereby puncturing the uterus, whereby said IUD is provided with anchoring means comprised of a knot and a noose;
removing the applicator from the uterus and threading the noose of the anchoring knot to the serosa with a biodegradable knot or by a metal or biodegradable clip.
